(19) United States
(12) Patent Application Publication
(10) Pub. No.: US 2007/0042970 A1
(43) Pub. Date: Feb. 22, 2007

(54) Folate-modified cholesterol-bearing pullulan as a drug carrier

(75) Inventors: Junzo Sunamoto, Kusatsu-shi (JP); Kazutoshi Ushio, Niihama-shi (JP); Masaaki Hidaka, Nagasaki-shi (JP)

Correspondence Address: OLIFF & BERRIDGE, PLC, P.O. BOX 19928, ALEXANDRIA, VA, 22320, US

(73) Assignee: CHEMICAL SOFT R&D INC., Kyoto-shi (JP)

(21) Appl. No.: 11/207,888

(22) Filed: Aug. 22, 2005

(51) Int. Cl. A61K 31/704; A61K 31/7076

(52) U.S. Cl. 514/26; 514/34; 536/6.1; 536/17.4

(57) ABSTRACT

Folate modified cholesterol-bearing pullulan (FA-CHP) was synthesized by the reaction of folic acid γ-2-aminoethylamide and 4-nitorophenyl chloroformate-activated cholesterol-bearing pullulan, wherein folate and pullulan are connected through a NH—CH₂—CH₂—NH group. Approximately 0.5-1 folates are connected per about 100 glycoside units of pullulan. Then, several combinations of FA-CHP, cholesterol-bearing pullulan (CHP) and doxorubicin (DOX) mixture were tested for cancer selective cytotoxicity. A mixture of FA-CHP, CHP and DOX of 1:4:0.02 (weight ratio) gave sharp and selective damage to cells of a human epidermoid cancer KB known as expressing a high level of folate receptor. The same mixture inhibited the growth of HuH7 cells, which is a human hepatocellular carcinoma and is unknown as a folate receptor.

The present invention relates to the synthesis of folate modified cholesterol-bearing pullulan(FA-CHP) and to a drug delivery system comprising folate modified cholesterol-bearing pullulan(FA-CHP) and an anti-cancer drug. More specifically, it relates to the synthesis of folate modified cholesterol-bearing pullulan(FA-CHP) which provides purer FA-CHP than former methods, and thus FA-CHP synthesized according to the present investigation should give clearer cytotoxicity than formerly. The FA-CHP nanoparticle comprising an anti-cancer drug showed cytotoxicity to cancer cells and was found to be useful drug carriers for cancer treatments.

### BACKGROUND OF THE INVENTION

Cancer is a serious illness, and its effective cure often requires patients with great endurance. To overcome the many difficult problems of cancer treatment, various chemotherapeutic reagents have been studied, and some of them have been applied for practical uses. However, in most cases, serious side effects have so far been unavoidable.

It is well known that not a few cancer cells express enhanced levels of folate receptor (Weitman, S. D., Lark, R. H., Coney, L. R., Fort, D. W., Frasca, V. Zurawski, Jr., V. R., and Kamen, B. A. Cancer Research 52, 3396-3401 (1992); Ross, J. F., Chaudhuri, P. K., and Ratnam, M. Cancer 73, 2432-2443 (1993); Prasad, P. D., Ramamoorthy, S., Moe, A. J., smith, C. H., Leibach, F. H., and Ganapathy, V. Biochim. Biophys. Acta 1223, 71-75 (1994); Li, P. Y, Vecchio, S. D., Fonti, R., carriero, M. V., Potena, M. I., Botti, G., Miotti, S., Lastoria, S., Menard, S., Colnaghi, M. I., and Salvatore, M. J. Nuclear Med. 37, 665-672 (1996); Antony, A. C. Annu. Rev. Nutr. 16, 501-521 (1996); Bueno, R., Appasani, K., Mercer, H., Lester, S., and Sugarbaker, D. J. Thoracic Cardio. Sur. 121, 225-233 (2001)). These facts have attracted considerable attention (Reddy, J. A. and Low, P. S. Critical Reviews in Therapeutic Drug Carrier Systems 15, 587-627 (1998); Drummond, D. C., Hong, K., Park, J. W., Benz, C. C., and Kirpotin, D. B. Vitamins and Hormones 60, 285-332 (2001); Sudimack, J. B. A. and Lee, R. J. Advanced Drug Delivery Reviews 41, 147-162 (2000)) and have been exploited for developing cancer selective drug delivery system (DDS) (Lee, R. J. and Low, P. S. J. Biol. Chem. 269, 3198-3204 (1994); Lee, R. J. and Low, P. S. Biochim. Biophys. Acta 1233, 134-144 (1995); Rui, Y., Wang, S., Low, P. S., and Thompson, D. H. J. Am. Chem. Soc. 120, 11213-11218 (1998); Goren, D., Horowits, A. T., Tzemach, D., Tarshish, M., Zalipsky, S., and Gabizon, A. Clinical Cancer Research 6, 1949-1957 (2000)). Cancer specific antigens can be exploited based on development of DDS with active targeting of cancer cells. Sunamoto, J. et al. have developed a nanosize hydrogel particle of hydrofobized polysaccharide as an efficient DDS, and cholesterol-bearing pullulan (CHP) and its derivatives have been applied to specific cancer targeting delivery (Akiyoshi, K. and Sunamoto, J. Supramolecular Science 3, 157-163 (1996); Ichinose, K., Yamamoto, M., Khoji, T., Ishii, N., Sunamoto, J., and Kanematsu, T. Anticancer Res. 18, 401-404 (1998); Matsukawa, S., Yamamoto, M., Ichinose, K., Ohata, N., Ishii, N., Kohji, T., Akiyoshi, K., Sunamoto, J., and Kamematsu, T. Anticancer Res. 2339-2344 (2000)).

### SUMMARY OF THE INVENTION

In the present invention, to develop another cancer-targeted DDS, folate modified cholesterol pullulan (FA-CHP), wherein the folate is connected to pullulan through a —NH—CH₂—CH₂—NH— group, was synthesized through the following steps as,
(i) Synthesizing pyrofolic acid through cyclic-amidation of folic acid,
(ii) synthesizing pteroyl hydrazide from pyrofolic acid and hydrazine,
(iii) synthesizing pteroyl azide from pteroyl hydrazide and t-butyl nitrile,
(iv) synthesizing folic acid γ-methyl ester from pteroyl azide and γ-methylglutamate,
(v) synthesizing folic acid γ-2-aminoethylamide (EDA-FA) from folic acid γ-methyl ester and ethylenediamine,
(vi) activating pullulan (CHP) by combining with 4-nitrophenyl chloroformate, and
(vii) synthesizing FA-CHP by combining EDA-FA with activated CHP.

The —NH—CH₂—CH₂—NH— group between the folate group and the pullulan allows a freedom of motion of the folate group to the large pullulan moiety, and thus the folate group appears to the surface of a nano-particle of the large pullulan moiety so that the complex formation of the folate group with another molecule is easily expected. The cholesterol group attached to the pullulan moiety enhances the complex formation of the folate group. Thus clearer medical effects can be obtained using the present FA-CHP.

This nanosized polymeric drug carrier composed of FA-CHP or a mixture of FA-CHP and CHP (FA-CHP-CHP) was then made a complex with doxorubicin(DOX), one representative anti-cancer drug.

Using a human cancer cell KB (TKG-4010), known as a good folate receptor (McHugh, M. and Cheng, Y. C. J. Biol. Chem. 254, 94-105 (1979); Antony, A. C., Kane, M. A., Portillo, R. M., Elwood, P. C., and Kolhouse, J. F. J. Biol. Chem. 260, 14911-14917 (1985)), selective damage of KB cells by FA-CHP-DOX or FA-CHP-CHP-DOX complexes (not attainable by free DOX of the same amount) was demonstrated.

FA-CHP-DOX or FA-CHP-CHP-DOX complexes also showed cytotoxicity toward HuH7 cells, a human hepatocellular carcinoma that is unknown as expressed folate receptor, but not toward HepG2 cells, a human hepatoblastoma carcinoma.

It has been shown that FA-CHP is an efficient drug carrier. Then, it can be used for better cancer therapy in the near future.

### BRIEF DESCRIPTION OF THE DRAWINGS

FIG. 1. Comparison of effects of FA-CHP-CHP-DOX with those of free DOX solutions on the growth of KB cells. Each 20 μl of the following solutions was added to cultivation medium after medium exchange. ◯; control; PBS, □; fc; FA-CHP (1 mg)+CHP (4 mg) in 1 ml PBS, Δ; d1; DOX 0.02 mg/ml-PBS, ▴; d2; DOX 0.03 mg/ml-PBS, ⋄; fcd 1; FA-CHP (1 mg )+CHP (4 mg)+DOX 0.02 mg in 1 ml PBS, ♦; fcd 2; FA-CHP (1 mg )+CHP (4 mg)+DOX 0.03 mg in 1 ml PBS.

FIG. 2. Cytotoxicity of CHP and FA-CHP toward KB cells in folate-depleted medium. control: PBS only.

FIG. 3. Cytotoxicity of a mixture of FA-CHP, CHP and DOX (1.00:4.00:0.02) toward KB cells in folate-depleted medium. control: PBS only, DOX: DOX (0.02 mg/ml), CD: a mixture of CHP and DOX (5.00:0.02), FCD: a mixture of FA-CHP, CHP and DOX (1.00:4.00:0.02).

FIG. 4. Cytotoxicity of a mixture of FA-CHP, CHP and DOX (1.00:4.00:0.02) toward HuH7 cells in folate-depleted medium. Notations are identical as before.

FIG. 5. Cytotoxicity of a mixture of FA-CHP, CHP and DOX (1.00:4.00:0.02) toward HepG2 cells in folate-depleted medium. Notations are identical as before.

FIG. 6. Cytotoxicity of FA-CHP, CHP and DOX (1.00:4.00:0.02) complexes in folated-depleted medium. Notations are identical as before.

### DETAILED DESCRIPTION OF A PREFERRED EMBODIMENT

### 1. Synthesis of Folic Acid-2-aminoethylamide (EDA-FA), 10.

EDA-FA was synthesized through the following five steps based on the procedure of Luo, J. et al. (1997) with slight modifications.

### Step 1: Cyclic-Amidation of Folic Acid, 3, Synthesis of Pyrofolic Acids, 4.

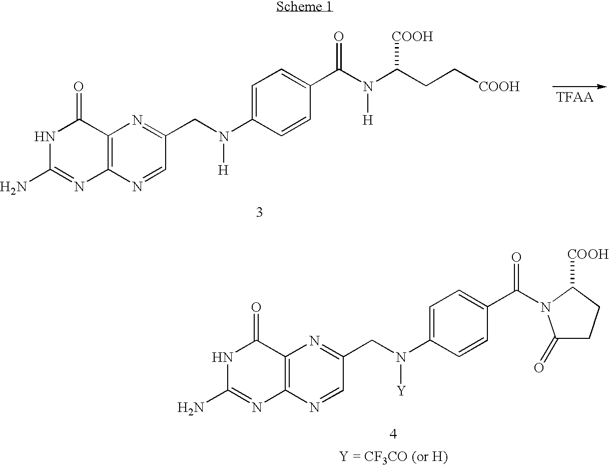

Folic acid, 3 (4.0 g, 9.2 mmol) was dissolved in dried THF (40 ml) and stirred by a magnetic stirrer on an ice-bath under nitrogen atmosphere. Since the folic acid did not dissolve very well in THF, the solution was cloudy orange. Trifluoroacetic anhydride (10.5 ml, 74 mmol) (TFAA) was slowly added using a syringe, and the solution was kept on the ice-bath for another 30 min.

The solution was then immersed in a water-bath (25° C.). The precipitate vanished gradually and the solution turned homogeneous in a clear dark-orange color. The solution was kept at 25° C. for 6 hours until all the folic acid was consumed.

The solution was filtrated through a celite pad and concentrated by a rotary evaporator to remove most solvent and excess trifluoroacetic anhydride. The residue was then transferred by the aid of 20 ml THF into a round bottom flask. Five grams of small ice pieces were added piece by piece to the solution. The solution was kept at room temperature for several hours until the reaction was completed.

The products were precipitated by slowly transferring the solution into 80 ml of diethyl ether. The precipitates were collected by filtration under reduced pressure, followed by washing three times with 20 ml of diethyl ether. The solid product was vacuum dried for 48 h, which gave 4.3 g of of N¹⁰-(trifluoroacetyl)pyrofolic acid, 4; yield 88%.

HPLC analysis of the product: 20 μl of the sample was dissolved in 1 ml of 10 mM potassium phosphate buffer (pH 7.0) and analyzed using an HPLC (Tosoh TSK-GEL ODS-80TS, 4.6×150 mm). The analysis was performed with gradient elution at a flow rate of 1.0 ml/min using acetonitrile as eluent A and 10 mM phosphate buffer with 3 MM NaN₃ (pH 7) as eluent B; gradient 0 min, 0% A: 2 5 min 40% A; sample of injection: 20 μl.

### Step 2: Synthesis of Pteroyl Hydrazide, 5

N¹⁰-(trifluoroacetyl)pyrofolic acid, 4, (4 g, 7.7 mmol) was fully dissolved in 100 ml of DMSO with stirring under nitrogen atmosphere. Three ml of hydrazine (anhydrous, 94 mmol) was slowly introduced using a pipette, and the reaction mixture was kept at 25° C. for 10 h.

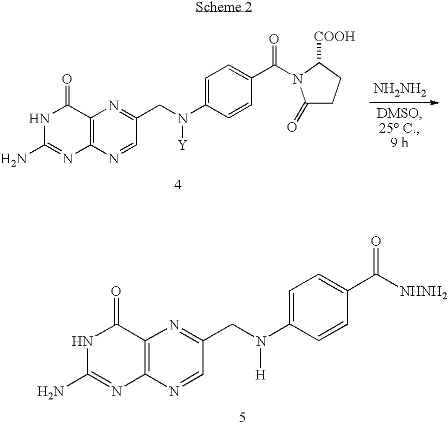

The reaction mixture was then filtered through a celite pad to remove trace solid, and 100 ml of methanol was slowly added into the filtrates while stirring, forming a yellowish precipitate. The precipitates were filtrated, washed 3 times with 20 ml methanol and two times with 20 ml ether, and then subjected to vacuum-drying for 24 hours, which gave 2.2 g of the product, pteroyl hydrazide, 5; yield 90%.

### Step 3: Synthesis of Pteroyl Azide, 6

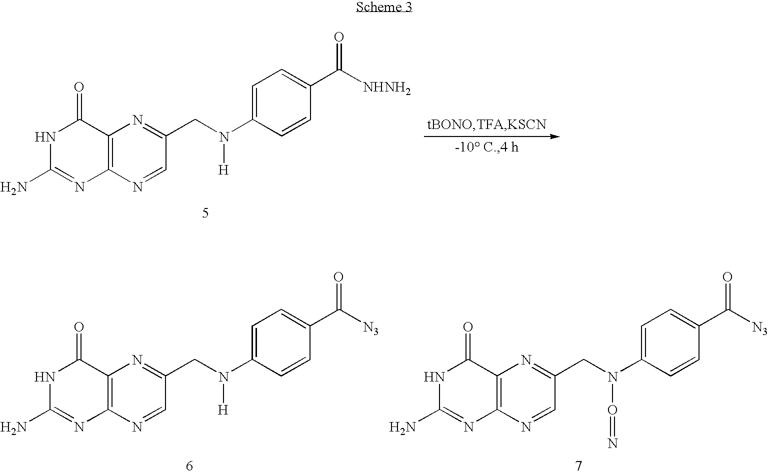

Ice-cold trifluoroacetic acid (20 ml) was introduced into a flask with 2.2 g (6.9 mmol) of pteroyl hydrazide, 5, and 32 mg of KSCN (potassium thiocyanate, 0.33 mmol). After the solid material was dissolved, the reaction mixture was cooled on a 2-propanol-dry ice bath, followed by slow addition of t-butyl nitrile (0.78 ml, 6.9 mmol). The reaction was carried out for 4 h, and then sodium azide (3.45 mmol) was added. The solution was brought back to room temperature for another hour, and the solution was filtered by a celite pad. The filtrates of the solution were slowly added 20 ml 2-propanol, and the precipitate was collected by centrifugation. The precipitate was subsequently washed by water (3×20 ml), acetonitrile (20 ml) and then diethyl ether (20 ml). The product was redissolved in 20 ml of trifluoroacetic acid and re-precipitated in the same way. The product, pteroyl azide, 6, was vacuum-dried and was 1.6 g; yield, 70%.

### Step 4: Synthesis of Folic Acid γ-methyl Ester, 9

Pteroyl azide, 6, (1.6 g, 4.7 mmol) and γ-methyl glutamate, 8, (0.84 g, 5.2 mmol) were dissolved in 20 ml of dried DMSO, followed by the addition of 1.2 ml (9.5 mmol) of tetramethylguanidine.

The solution was stirred for about 6 h at room temperature, then filtered by a celite pad, and precipitated by slowly adding 130 ml of acetone. The crude precipitate was washed by 2 portions of diethylether (50 ml) and vacuum-dried, which gave 1.9 g of the product; yield, 90%.

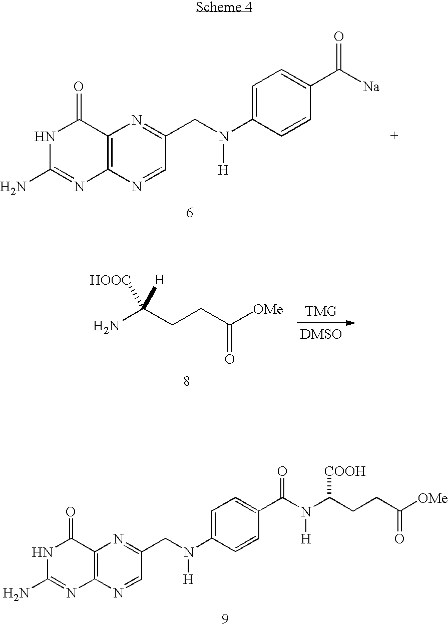

### Step 5: Synthesis of Folic Acid γ-2-aminoethylamide (EDA-FA), 10

Ethylenediamine (14 ml) was added directly to tetramethylguanidinium 1-methyl folate, 9, (1.9 g, 4.2 mmol) at the room temperature. The mixture was transfered into 300 ml of a mixture of acetonitrile/diethyl ether, 1:1 by volumn. The precipitate was collected by centrifugation and re-dissolved in 300 ml Milli-Q water. The pH of the aqueous solution (clear-dark orange solution) was adjusted to 7.0 by 5% HCl. The solid materials were collected by centrifugation, wash by water (250 ml×3), acetonitrile (250 ml×2), and diethyl ether (100 ml×2). The yield of the final product, 10, was 1.4 g after and vacuum dried; yield, 72%

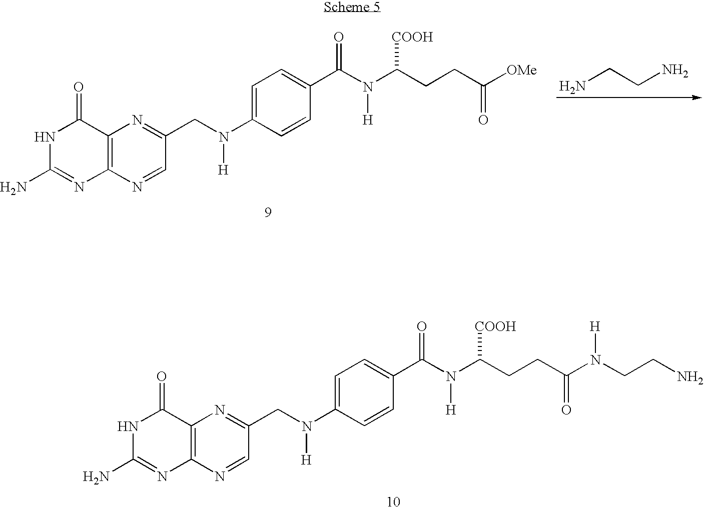

### Step 6: Purification of EDA-FA

The crude EDA-FA (1 g) was added into 40 ml of milli-Q water, and the solution was titrated by 0.1 M NaOH to pH 11 until the entire solid dissolved. The obtained solution was centrifuged to completely remove undissolved materials, then submitted to a preparative HPLC, Tosoh TSK-GEL ODS-80TS, 215×300 mm with a gradient elution at the flow rate 4.0 ml/min; eluent A: acetonitrile; eluent B: 10 mM; sample of injection: 4 ml. The collected fractions were lyophilized, redissolved in Milli-Q water (pH 11) and precipitated at pH 7. The precipitates were washed with Milli-Q water three times and finally vacuum-dried. 430 mg of purified EDA-FA was collected; yield, 43%.

### 2. Synthesis of Folic Acid Modified-CHP (FA-CHP), 1

FA-CHP was synthesized through activation of CHP and conjugation of EDA-FA to activated CHP.

### (1) Activation of CHP by 4-Nitrophenyl Chloroformate; Synthesis of 11 (See Scheme 6)

Before conjugating EDA-FA, 10, to CHP, 2 (CHP used in this study was CHP-108-0.9), CHP was activated by 4-nitrophenyl chloroformate (PNPC). Since the conjugation ratio of folic acid moiety to CHP largely depends on the molar ratio of reactants (chloroformate, EDA-FA and CHP), the reaction time, and especially the presence of trace water, it would be advisable to use an almost comparable amount of PNPC to a glycoside unit of CHP and to use a slightly larger amount of PNPC-activated CHP for the conjugation between CHP and EDA-FA.

CHP, 2, (cholesterol-bearing pullulan, CHP-108-0.9, Medical Purpose Grade, Nippon oil & fat Co. LTD., Tokyo, Lot 000125) (400 mg, 2.39 mmol as the glycoside unit) was pre-dissolved in 18 ml of dried DMSO/pyridine, and the solution was continually stirred on an ice-bath. PNPC (497 mg, 2.40 mmol) was pre-dissolved in 1 ml of 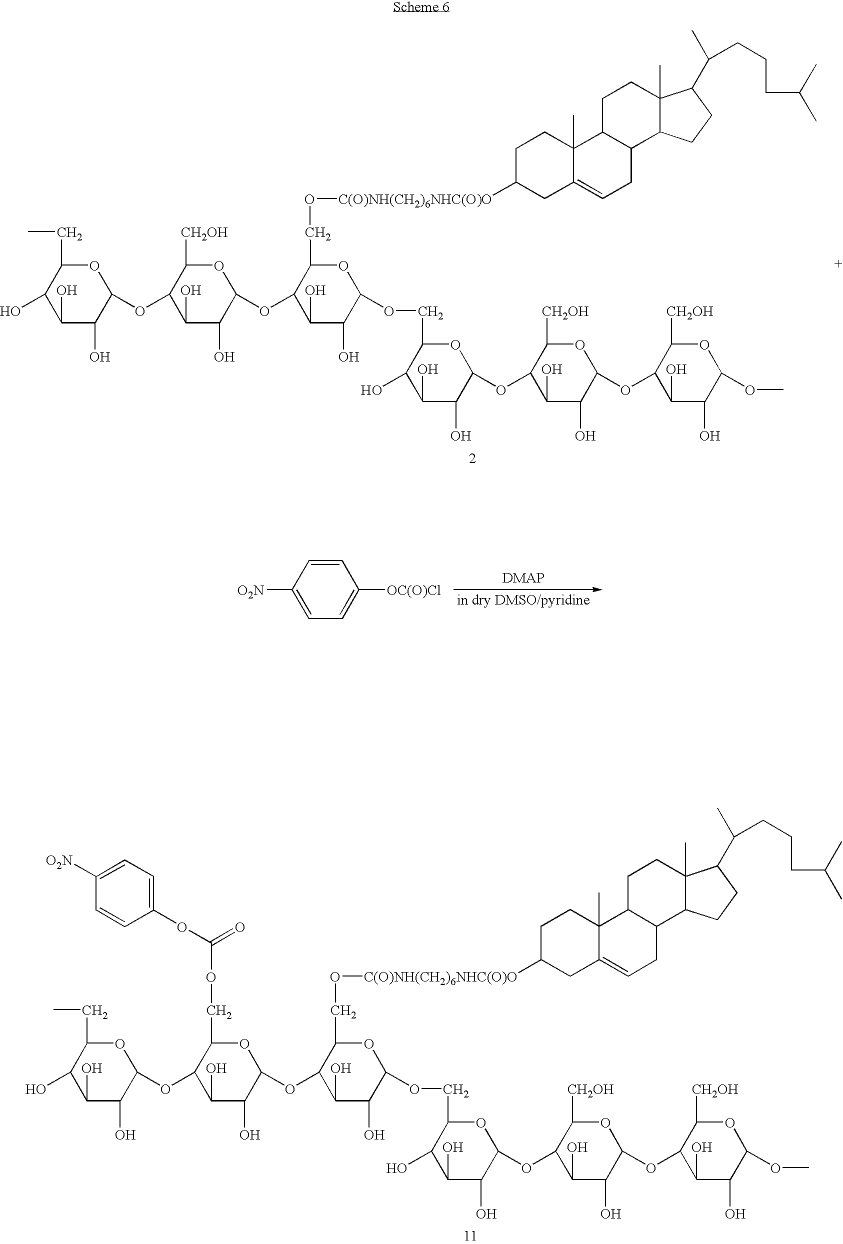
DMSO/pyridine and slowly introduced using a syringe to CHP solution, followed by the addition of DMAP (24 mg, 0.20 mmol) dissolved in 1 ml of pyridine. The solution was stirred for 3 h in the dark on an ice-bath, and then brought back to the room temperature for another hour. The reaction was quenched by addition of 30 ml of anhydrous ethanol. Precipitates were collected by centrifugation, washed by ethanol (3×30 ml), and were subject to vacuum drying overnight. The obtained activated CHP, 11, was 433 mg (2.13 mmol of glycoside units); yield, 90%.

Three mg of activated CHP was dissolved in 3 ml of 0.1 M NaOH solution, and the content of PNPC was determined by the absorption at 400 nm (molar extinction coefficient ε⁴⁰⁰=18,400 M⁻¹ cm⁻¹).

### (2) Synthesis of FA-CHP, 1 (See Scheme 7)

The activated CHP 11 (300 mg, 1.47 mmol as glycoside unit) was dissolved in 40 ml of dried DMSO/pyridine (1:1 v/v), and the solution was stirred for 4 h for complete dissolution at room temperature. EDA-FA, 10, (141.8 mg, 0.29 mmol ) was then added into the solution, followed by the addition of 20 mg DMAP dissolved in 1 ml of pyridine. The solution was stirred at room temperature for three days in the dark.

The reaction was quenched by addition of 30 ml of ethanol, and the solution was continually stirred overnight on an ice-bath. Pale-yellowish precipitates were collected by centrifugation, washed by anhydrous ethanol (30 ml×2). The precipitates were re-dissolved in 40 ml of DMSO, then transferred to a dialysis bag and dialyzed against 200 ml of 0.01 M NaOH solution. The dialysis was repeated for 4 days (every 4-hours, 5 exchanges a day). The solution was assayed everyday by HPSEC chromatography to monitor the solution behavior. Three mg of activated CHP in 3 ml DMSO was dialyzed in the same way as described above to monitor progress of hydrolysis of PNPC. The hydrolysis reaction was completed in 4-day-dialysis.

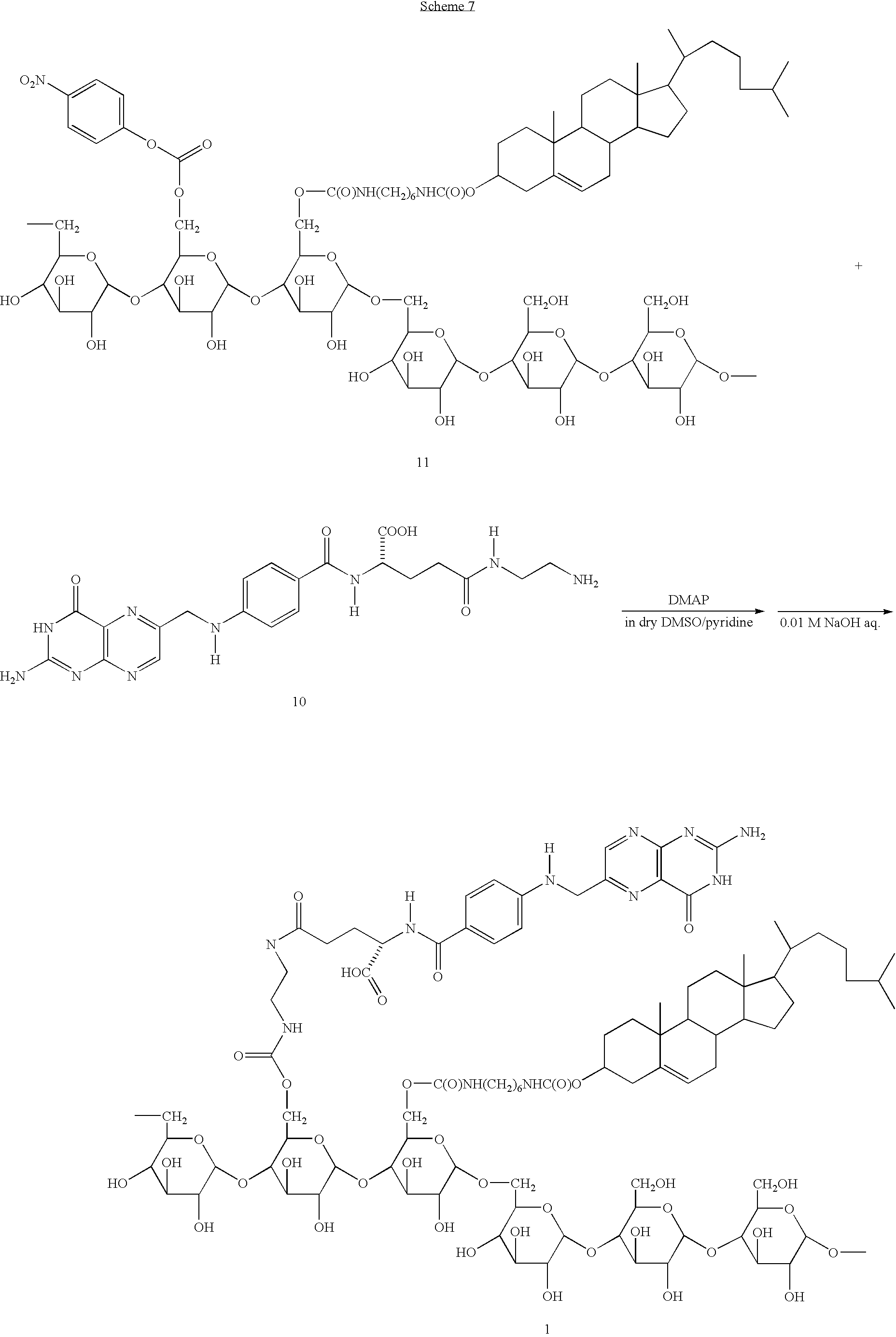

In the preparation of FA-CHP, for all the intermediates the separation and purification were very hard and difficult because of their weak solubility in solvents. Preparative column chromatography was very useful and powerful technique.

### (3) Purification of FA-CHP

The dialyzed solution of FA-CHP, which contained crude FA-CHP, was titrated to pH 7 by addition of 5% HCl. The sample was purified by Sephacryl S-500 column (20×430 mm) chromatography as eluted by potassium phosphate buffer (10 mM, pH 7 in the presence of 3 mM NaN₃) at the flow rate of 1.0 min/ml. All the fractions from the chromatography were collected and assayed by HPSEC. HPSEC analysis was carried out in Tosoh GX-4000 column at the flow rate of 0.5 ml/min using 10 mM phosphate buffer (pH 7) containing 3 mM NaN₃ as eluent, and the chromatogram was monitored by a Tosoh RI-8020 reflect index detector and a Waters 991 Photodiode array. The fractions containing FA-CHP collected were again dialyzed against 1 L of Milli-Q water for 24 h (6 exchanges), then were subjected to freeze-drying. Pale-yellowish product, 1, was obtained (170 mg). The polysaccharide content was determined by the phenol-sulfuric acid assay, and the content of folate moiety (FA) was measured by UV at 363 nm. The mass percentage of FA in FA-CHP was determined as 28%, and the substitution ratio was 10% (10 FA out of 100 glycoside units).

Purity of the intermediates and the final product were investigated by mostly 500 MHz ¹H-NMR, FFT-IR and HPLC.

### (4) The Ratio of Substitution

The most desirable substitution ratio of the folate moiety was 0.5-1% and that of the cholesterol moiety was also 0.5-1%, where 1% indicates one substituent out of 100 glycoside units. This finding indicates that both moieties could not be connected to a specific glycoside unit probably because of steric hindrance.

It is possible to have a substitution ratio larger or smaller than the above value and the product is still useful as a drug carrier. We have synthesized FA-CHP in which the substitution ratio of the folate moiety was 4.5%. This FA-CHP (4.5%) was only as good as FA-CHP (1%) as a drug carrier, though the synthesis was much harder. Thus, there are an upper limit and a lower limit of the desirable substitution ratio. If the ratio is much smaller than the above value, the ability to form a complex with an anti-cancer drug is too small and such FA-CHP is not useful. If the ratio is larger, the synthesis becomes very difficult, the purity of the product would be lower, and such FA-CHP would not show a remarkable effect as expected by the high substitution ratio.

### 3. Use of FA-CHP as a Drug Carrier

FA-CHP prepared in the present invention is useful as a drug carrier because cholesterol is connected to pullulan and the folate group is connected to pullulan through a —NH—CH2-CH2-NH— group. Because of the hydrophobic character of cholesterol FA-CHP forms a nano particle in water. Because of the hydrophilic character of the folate group and the flexibility of the —NH—CH2-CH2-NH— group the folate group can stay on the outer surface of the nano particle.

The present invention will be explained more specifically by referring to the following examples. However, the scope of the present invention is not limited to the following examples.

### EXAMPLE 1

### Cancer Cell Selective Cytotoxicity of FA-CHP-DOX and FA-CHP-CHP-DOX Nanoparticle Solutions

(1) Preparation of FA-CHP-DOX and FA-CHP-CHP-DOX Complexes.

In order to control the surface density of the folate moiety on CHP hydrogel nanoparticle, FA-CHP and CHP were mixed for the complex formation. A mixture of FA-CHP (1.0 mg) and CHP (4.0 mg) was dissolved in 0.7 ml of PBS (polybutylene succinate: Nissui Phamaceutical Co.) by standing the mixture overnight at 37° C.

Doxorubicin(DOX), one representative anti-cancer drug, was a gift from Dr. K. Ichinose, Nagasaki Univ. Then, 0.1 g/L aqueous DOX solution was separately prepared by dissolving DOX in PBS and stocked at −20° C. before use.

Then, 0.3 ml (or 0.2 ml +0.1 ml PBS etc.) of the DOX stock solution was added to the FA-CHP-CHP mixed solution and incubated for 15 h at 37° C. The mixture was directly used after filtration (0.2 μm) for sterilization in the experiments described in this report.

FA-CHP was easily soluble in PBS, and FA-CHP-DOX complex was prepared similarly. For example, 5.0 mg of FA-CHP can simply complex with 0.01 mg of DOX. Complexes of DOX with a mixture of FA-CHP and CHP are denoted as FA-CHP-CHP-DOX hereafter.

(2) Cells

A human epidermoid carcinoma KB (TKG-4010) was obtained from Cell Resource Center of Tohoku University. A cell line of KB was cultured in folate-depleted Dulbecco's modified Eagle's medium (DMEM) supplemented with 10% heat-inactivated fetal bovine serum, penicillin (50 units/ml), streptomycin (50 μg/ml), 1.5 mM Hepes in a humidified atmosphere containing 5% CO₂ at 37° C. Folate-depleted DMEM was prepared in our laboratory basically according to the composition of DMEM described by Nissui Pharm. Co. only without the addition of folic acid. A human fetal lung fibroblast TIG-1-20 (JCRB-0501) was obtained from JCRB cell bank, and used as a control normal cell. TIG-1-20 was also cultured in the same medium in the present experiments. For maintaining culture, the cells were cultured in 5 ml of folate-depleted DMEM in 5 cm plastic dishes.

Cells of both KB and TIG-1-20 was co-plated in 6 well cultivation plate at the cell density of each ca. 3×10⁴/ml and preincubated for 2 days and the medium folate-depleted DMEM of each well was replaced with a new one before the addition of reagents. Drug mixtures tested were added with 1V/V % of the culture medium. Then, the growth and morphologic feature of the cells were microscopically recorded once a day for at least 4 days

Cells of either KB or TIG-1-20 were plated in 6 well cultivation plates and preincubated for 2-3 days, and each cultivation medium was replaced with new one (1.98 ml) just before the addition of anticancer reagents (0.02 ml). At the time of the addition, cell densities were about 5×10⁴/ml for TIG-1-20, and about 15×10⁴/ml for KB (each ca. 20-25% of their confluent states). The additives mainly used were; Control 1: PBS, Control 2 (FC): FA-CHP (1 mg)-CHP (4 mg) complex solution in 1 ml PBS, DOX 1: 0.02 mg doxorubicin/ml-PBS, DOX 2: 0.03 mg doxorubicin/ml-PBS, FA-CHP-CHP-DOX 1 (FCD 1): FA-CHP (1 mg)-CHP (4 mg)-DOX (0.02 mg) in 1 ml PBS, FA-CHP-CHP-DOX 2 (FCD 2): FA-CHP (1 mg)-CHP (4 mg)-DOX (0.03 mg) in 1 ml PBS. Drug mixtures with other composition were also tested essentially by the same procedure.

The culture medium was removed to 15 ml centrifugation tube, and cells remained attached to the dish were removed with Sigma cell dissociation solution (non-enzymatic, in PBS) in the case of KB, and with 0.05% trypsin and 0.02% EDTA solution in the case of TIG-1-20. Each cell in a well was treated by 0.5 ml of the corresponding dissociation solution for 10-15 min, and after pipetting 1.5 ml of the cultivation medium folate-depleted DMEM was added and suspended again by pipetting. 0.2 ml of the cell suspension was transferred to an eppendolf tube and 20 μl of 0.5% trypan blue solution (in PBS) was added. After mixing with pipetting, all cells were counted on improved Neubauer hematocytometer. Detached cells were also counted by the same procedure after adjusting the removed medium to 2 ml.

(3) Cytotoxicity of FA-CHP-CHP-DOX Complex.

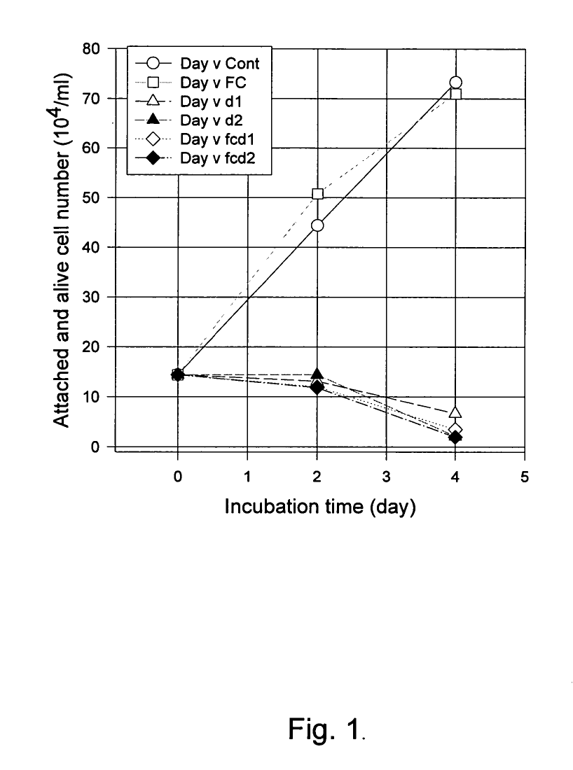

As the first screening of complexed mixtures for cancer selective toxicity, we investigated FA-CHP-DOX and FA-CHP-CHP-DOX mixtures using KB and TIG-1-20 mixed cell culture. Either FA-CHP-DOX and FA-CHP-CHP-DOX, mixtures with the ratio ≧0.05:5 (DOX:FA-CHP or FA-CHP-CHP) seemed to be rather toxic not only for KB but also for TIG-1-20. On the other hand, complexed mixtures with the weight ratio 0.01-0.03:5 (DOX:FA-CHP or FA-CHP-CHP) looked effective and selective against cancer KB cells. Representative data (FA-CHP-CHP-DOX 1:4:0.02 and 0.03 case) of microscopic observation were shown in FIG. 1. The presented data are those at 4 days after the addition of drug mixtures.

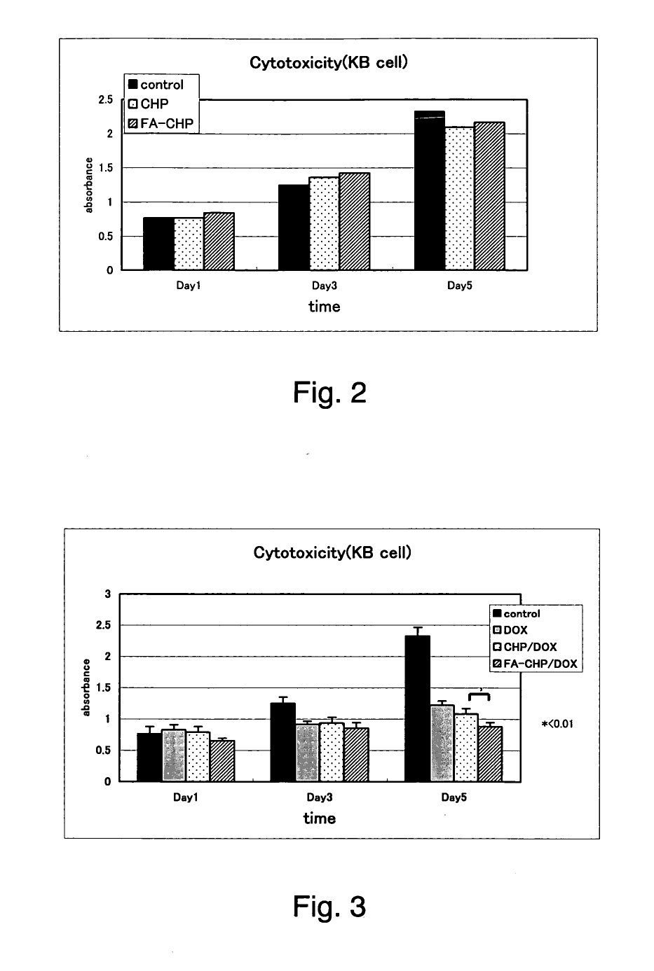

Because of the hopeful observation in the previous section, we proceeded to cell counting experiments with either KB or TIG-1-20 cells, using several FA-CHP-CHP-DOX complexes. Among them, FA-CHP-CHP-DOX (weight ratio: 1:4:0.02) gave rather good results (FIG. 2-5).

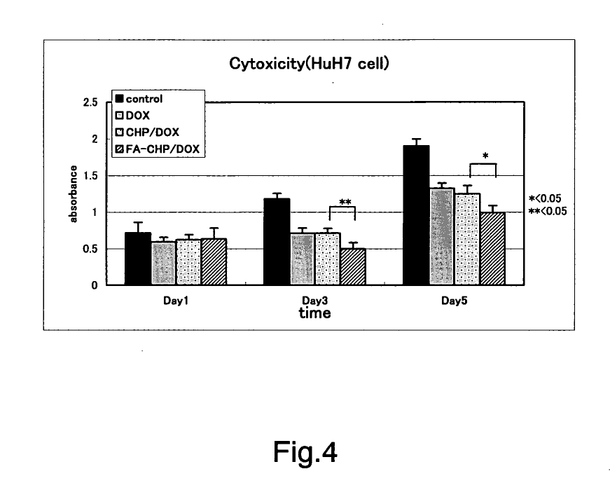

As shown in FIG. 2 (cell number counting) and FIG. 3 (morphology under a phase contrast microscopy) in KB case, FA-CHP-CHP-DOX complex severely inhibited the cell growth and cells were led to apoptosis. The effectiveness of the complex might be even superior to free DOX, although, of course, experiments with more diluted additives must be carried out. The morphological features (FIG. 4) well coincided with the results obtained by cell counting experiments. Particularly notable feature is that the number of KB cells survive in the presence of FA-CHP-CHP-DOX (1:4:0.02) (0.2 mg/L DOX) seemed to be smaller compared to that in the presence of a corresponding amount of free DOX (0.2 mg/L).

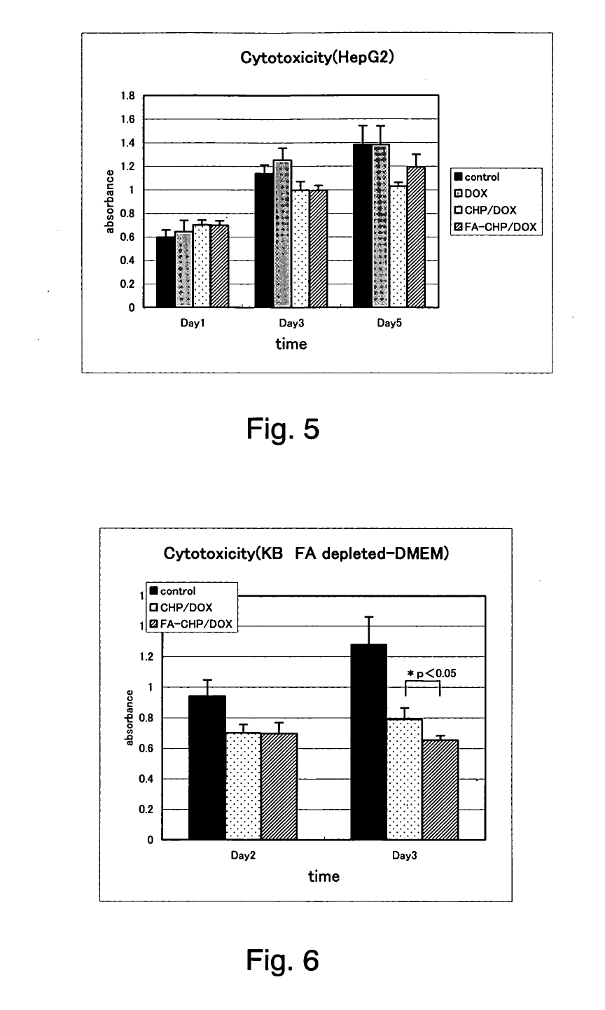

In the case of TIG-1-20, however, FA-CHP-CHP-DOX (1:4:0.02) dramatically prevented the cell death at least for 2 days contrary to the case of KB, while the corresponding free DOX (0.2 mg/L) completely killed cell proliferation (FIG. 4). Microscopic photographs of TIG-1-20 at 4 days after the addition of reagents seemed to coincide well with the results of cell number counting (FIG. 5).

As shown in FIG. 4, the TIG-1-20 cell grows better in the case of FA-CHP-CHP-DOX (1:4:0.02) than in the case of PBS control. This finding shows not only the excessive deficiency of folic acid for TIG 1-20 cells in folate depleted DMEM medium but also activation of the vital growth of TIG-1-20 by the FA-CHP-CHP added.

### EXAMPLE 2

(1) Materials and Methods

Doxorubicin (DOX) was the gift from Kyowa Hakko Kogyo Co. Japan. CHP (cholesterol-bearing pullulan, CHP-108-0.9, Lot 000125) was the gift from Nippon oil & fat Co. LTD., Tokyo.

KB cells (TKG0401), a human epidermoid carcinoma that overexpressed the folate receptor, was obtained from Cell Resource Center for Biomedical Research Institute of Development, Tohoku University. In in vitro studies KB cells were cultured in folate depleted DMEM supplemented with 10% heat-inachtivated fatal bovine serum (FBS)(GIBCO), penicillin (100 unit/L), streptomycin (100 μg/L), amphotericin B (125 μg/L)(GIBCO), 1.5 mM HEPES, in a humidified atmosphere containing 5% CO2 at 37° C. HuH-7, a human hepatocellular carcinoma, and HepG2, a human hepatoblastoma, that unknown as expressed folate rceptor, were given to us by Dr. Nakao, Health Research Center, Nagasaki University. Both of them are unknown as expressed folate receptors. Folate depleted DMEM was prepared according to the composition of Nissui pharmaceutical Co. only without an addition of folic acid. KB, HuH7 and HepG2 cells were cultured in DMEM with 10% FBS, penicillin, streptomycin and flluconazol.

As the first step, 0.1 mg/ml DOX in PBS solution was prepared and stocked in −20° C. before use. FA-CHP (1 mg) and CHP (4 mg) were added to 0.2 ml of the above DOX solution, and the mixture was capped and incubated overnight at room temperature. Then, 0.8 ml PBS was added to the mixture and further incubated at 37° C. overnight with mild shaking. This formulation of the mixture of FA-CHP, CHP and DOX (weight ratio: 1.00:4.00:0.02) (denoted as FCD) was used directly after filtration (0.2 μm).

The MTT assay is a colorimetric assay based on the ability of viable cells to reduce a soluble yellow tetrazolium salt (MTT) to blue formazan crystals. Cell lines (KB, HuH7, HepG2) were seeded into 96 well plates at the various time after the agents (0.4% MTT dye solution and 0.1 M sodium succinate (1:1) solution) added, and the plates were incubated for 3 hours in a humidified chamber at 37° C. The medium in plates was aspirated, the dye was eluted with dimethylsulfoxide (DMSO) and 2N KOH(1:1), and the absorbance was measured at 450 nm.

(2) Cytotoxicity of FA-CHP and CHP

KB, HuH7 and HepG2 were seeded into 96 well plates at the cell density 2×10⁴/ml during 24 hr before the addition of agent in folate-depleted DMEM. Next day the medium was changed, and the agent (2 μl) in 1% of the medium (200 μl) was added. The following additives were used; control (PBS, denoted as cont), DOX(0.02 mg/ml), CHP-DOX (weight ratio, 5.00:0.02 mg/ml, denoted as CD), FA-CHP, CHP and DOX (weight ratio, 1.00:4.00:0.02, denoted as FCD). The MTT assay was performed at day 1, 3 and 5 after the addition of the agent.

KB cells were seeded in folate-depleted-DMEM and folate-overdosed-DMEM at the cell density 2×10⁴/ml before the addition of agent. The medium was changed the next day, and the agent (20 μl) in 10% of the folate deplete and overdosed medium (200 μl) was added. The cells were incubated for 3 hours after the addition of the agent, and then the medium was removed. The cells were washed twice by PBS and the medium was changed to DMEM. The MTT assay was performed at the day 2 and 3 after the addition of the agent.

(3) Selective Cytotoxicity of FA-CHP, CHP, DOX Complexes Toward Cancer Cell Lines.

CHP and FA-CHP did not have cytotoxicity toward cancer cells for itself as shown in FIG. 2.

The mixture of FA-CHP, CHP and DOX (weight ratio, 1.00:4.00:0.02) significantly inhibited the growth of KB cells rather than free DOX and the mixture of CHP and DOX at the day 5 in folate-depleted DMEM as shown in FIG. 3.

Similarly, as shown in FIG. 4, the same mixture of FA-CHP, CHP and DOX more significantly inhibited the growth of HuH7 cells than free DOX and the mixture of CHP and DOX at the day 3 and 5 in folate-depleted DMEM.

On the other hand, as shown in FIG. 5, the same mixture of FA-CHP, CHP and DOX was unable to inhibit the growth of HepG2 cells in folate-depleted DMEM. These data show that the mixture (FCD) selectively inhibited the growth of the KB cells (folate receptor) and HuH7 cells (unknown as folate receptor). Therefore, a folate receptor may be observed in HuH7 cells, but not HepG2 cells.

It is well-known that the cellular uptake of folate-PEG-liposomes was saturable and could be blocked with high concentration of free folic acid. In order to evaluate the effectiveness of cytotoxicity of the proposed formulation via folate receptor, the cytotoxicity of the same drug formulation toward KB cells (FR overexpressed cells) in folate-depleted and overdosed DMEM was performed.

The formulation of a mixture of CD and FCD was added separately for 3 hours into folate-depleted medium, and then the medium was removed, washed twice by PBS and replaced with new DMEM. Compared with the cytotoxicity of CHP as shown in FIG. 6, a mixture of FCD was significantly effective to inhibit the growth of KB cells at the day 3 in folate-depleted medium. On the other hand, the cytotoxicity of FCD was reduced in folate-overdosed medium. Accordingly, this selective cytotoxicity was probably mediated by the folate receptor.

Folic-acid conjugated cholesterol pullulan (FA-CHP) for itself has no cytotoxicity to inhibit the growth of cancer cells according to FIG. 2. Then, FA-CHP can be used as a vehicle for drug delivery. The formulation of a mixture of FA-CHP, CHP and DOX significantly inhibited the growth of both KB and HuH7 cells in folate-depleted DMEM. However, the same formulation didn't inhibit the growth of HepG2 cells in the same medium.

The cytotoxicity of this drug formulation toward KB cells was reduced in folate-overdosed medium. Accordingly, the selective cytotoxicity using the proposed formation was probably mediated by the folate receptor. This new vehicle may reduce the dose of anti-cancer drug and the side effects of chemotherapy.

What is claimed is:

1. Folate modified cholesterol-bearing pullulan (FA-CHP) wherein the folate and the pullulan are connected through a —NH—CH₂—CH₂—NH— group.

2. FA-CHP wherein the folate and the pullulan are connected through a —NH—CH₂—CH₂—NH— group, wherein the ratio of the folate group to the glycoside unit of the pullulan is 0.005-0.01.

3. A process for preparing FA-CHP wherein the folate and the pullulan are connected through a —NH—CH₂—CH₂—NH— group, comprising steps of:
(i) synthesizing pyrofolic acid through cyclic-amidation of folic acid;
(ii) synthesizing pteroyl hydrazide from pyrofolic acid and hydrazine;
(iii) synthesizing pteroyl azide from pteroyl hydrazide and t-butyl nitrile;
(iv) synthesizing folic acid γ-methyl ester from pteroyl azide and γ-methylglutamate;
(v) synthesizing folic acid γ-2-aminoethylamide (EDA-FA) from folic acid γ-methyl ester and ethylenediamine;
(vi) activating pullulan (CHP) by combining with 4-nitrophenyl chloroformate; and
(vii) synthesizing FA-CHP by combining EDA-FA with activated CHP.

4. A drug carrier in a drug delivery system for cancer selective cytotoxicity using FA-CHP, wherein the folate and the pullulan in the FA-CHP are connected through a —NH—CH₂—CH₂—NH— group.

5. A drug carrier in a drug delivery system for cancer selective cytotoxicity using a mixture of FA-CHP and cholesterol-bearing pullulan (CHP), wherein the folate and the pullulan in the FA-CHP are connected through a —NH—CH₂—CH₂—NH— group.

6. A drug complex of doxorubicin (DOX) as an anti-cancer drug and FA-CHP as a drug carrier for cancer selective cytotoxicity to KB cells and to HuH7 cells, wherein the folate and the pullulan in the FA-CHP are connected through a —NH—CH₂—CH₂—NH— group.

7. A drug complex of doxorubicin (DOX) as an anti-cancer drug and a mixture of FA-CHP and CHP as a drug carrier for cancer selective cytotoxicity to KB cells and to HuH7 cells, wherein the folate and the pullulan in the FA-CHP are connected through a —NH—CH₂—CH₂—NH— group.

8. A drug complex of doxorubicin (DOX) as an anti-cancer drug and FA-CHP as a drug carrier for cancer selective cytotoxicity to KB cells and to HuH7 cells, wherein the folate and the pullulan in the FA-CHP are connected through a —NH—CH₂—CH₂—NH— group and wherein the ratio of FA-CHP:CHP:DOX is substantially 1:4:0.02.
